(19) United States
(12) Patent Application Publication
(10) Pub. No.: US 2011/0004293 A1
(43) Pub. Date: Jan. 6, 2011

(54) ENDOVASCULAR PLATFORMS FOR THE DIFFERENTIAL TARGETING OF MOLECULES TO VESSEL WALL AND VESSEL LUMEN

(76) Inventors: Kumaran Kolandaivelu, Newton, MA (US); Abraham Tzafriri, Lexington, MA (US); Vijaya B. Kolachalama, Cambridge, MA (US); Elazer R. Edelman, Brookline, MA (US)

Correspondence Address: Matthew E. Connors, Esq.;Gauthier & Connors LLP, Suite 2300, 225 Fanklin Street, Boston, MA, 02110, US

(21) Appl. No.: 12/834,470

(22) Filed: Jul. 12, 2010

Related U.S. Application Data
(63) Continuation of application No. PCT/US2009/030712, filed on Jan. 12, 2009.
(60) Provisional application No. 61/010,724, filed on Jan. 11, 2008.

(51) Int. Cl. A61F 2/82

(52) U.S. Cl. 623/1.16; 623/1.42

(57) ABSTRACT

A drug delivering medical system intended for placement into a blood vessel is provided. The drug delivering medical system includes a stent device having a plurality of interconnected distinct strut elements comprising distinct strut element surfaces. At least some of the distinct strut surfaces are neither in contact with the lumen wall nor in contact with wall-contacting flow recirculation zones. The stent device releases at least one biologically active compound intended for distal delivery and provides sufficient surface area for delivering the required drug dose to a distal tissue.

This application claims priority from provisional application Ser. No. 61/010,724 filed Jan. 11, 2008, which is incorporated herein by reference in its entirety.

### BACKGROUND OF THE INVENTION

The invention is related to the field of endovascular devices, and in particular to an endovascular device that provides targeted delivery of therapeutic agents to local and/or regional targets through spatial patterning of drug release into specifically designed convective patterns established by the device. The devices and methods disclosed herein allow for the optimization of drug targeting to tissues that are distal to the sites of device implantation.

Coronary artery disease (CAD) is one of the leading causes of morbidity and mortality worldwide, leading to over 100,000 deaths annually in the United States alone. It is often thought of as a disease that results in progressive narrowing and/or acute occlusion of the vessels traversing the surface of the heart or epicardium. A multitude of therapies are aimed at relieving blockages of blood flow in these epicardial vessels. Their inherent proximal nature and relative large diameter makes these vessels amenable to percutaneous catheter based approaches such as angioplasty and stenting. While such interventions form a cornerstone of modern cardiovascular therapies, they fail to address the distal coronary vasculature where vessels taper to form smaller penetrating branches and downstream microvasculature. This distal vasculature forms a well recognized though under-addressed region of natural and iatrogenic disease. Moreover it is this crucial end location where oxygen and nutrient exchange takes place to allow viable myocardium making it an inherently high-impact region to consider when developing and delivering therapies to treat diseases intrinsic to the heart muscle.

The importance of the distal vasculature to atherosclerotic burden is becoming increasingly apparent. Over a lifetime, atherosclerosis results in extensive, wide-spread arterial narrowing. While it initially has a predilection to certain locations such as vessel bifurcation and regions of high tortuosity and curvature, it extends to create diffuse pathology that pays little heed to anatomic and geometric location. Populations such as diabetics and end stage renal patients who are at greatly increased risk for developing CAD exhibit an accelerated progression to diffuse states of disease. They often present without a particular lesion which can be targeted for treatment. In other populations such as women and African Americans, the pattern of disease is in fact skewed towards the more distal microvasculature. In its extreme, regional myocardial ischemic and infarction can occur without overt CAD observable during diagnostic catheterization (‘clean coronaries’) and is attributable to a more pure microvascular pathology in a condition known as cardiac Syndrome X. The tremendous potential impact of the distal circulation on affecting the pathogenesis of CAD is underscored in chronic disease conditions, where extensive collateralization can provide sufficient blood flow to sustain viable myocardium in otherwise unvascularized territories.

Iatrogenic processes can also result in distinct proximal and distal vessel pathology. Stents are load-bearing constructs commonly expanded at the site of epicardial stenosis to relieve the blockage: Following implantation, a slew of untoward biological reactions occur. These include well-studied local phenomena such as in-stent thrombosis and in-stent restenosis as well as uniquely distal phenomena such as the embolic shedding of atherosclerotic/thrombotic debris into the tapering downstream vasculature.

The inherent proximity between a predictable in-stent biological response and an implanted device has resulted in the logical application of local drug delivery which locally delivers anti-thrombotic and anti-proliferative molecules to curtail in-stent responses. However, even following successful and sustained relief of obstruction, coronary flow can be severely limited by distal disease and embolization. Various attempts have been made to address such states of ‘no-reflow’ generated by elevated distal flow resistance. Distal coronary capture devices are placed downstream of high-risk interventions to capture the shed debris that accompanies manipulation of highly diseased vessels.

However, given the highly bifurcating and tapered nature of the coronary bed, these filters are often insufficient and overly bulky to effectively capture shed debris and indeed, they have failed to show clinical benefit. Alternatively, selective intracoronary injection of vasoactive substances which counter vasospasm or anti-thromobtic/fibrinolytic agents aimed at breaking up emboli have been used to relieve downstream resistance and augment coronary flow. While these are effective in the immediate post-procedural setting and have been shown to improve both subjective symptoms and objective markers of coronary flow and heart function, their effects are short lived and are limited to the peri-interventional setting. Thus, standard drug eluting endovascular stents target drugs to the local, in-stent vicinity; methods to differentially target drugs to the distal vasculature and myocardium would have great potential and value.

Prior art has addressed the issue of local and downstream delivery. US Patent Pub. No. 2004/0142014 describes a stent device that offers a method of delivering agents to both the vessel wall and free stream via mural or luminal release respectively. What is not addressed in the prior art is how to optimize delivery to the luminal versus the mural side of the stent. While it is largely assumed that drug release beneath the strut will partition to the wall and drug released to the lumen will partition to the free stream, it is known that drug targeting to be far more complex.

### SUMMARY OF THE INVENTION

According to one aspect of the invention, there is provided a drug delivering medical system intended for placement into a blood vessel. The drug delivering medical system includes a stent device having a plurality of interconnected distinct strut elements comprising distinct strut element surfaces. At least some of the distinct strut surfaces are neither in contact with the lumen wall nor in contact with wall-contacting flow recirculation zones. The stent device releases at least one biologically active compound intended for distal delivery and provides sufficient surface area for delivering the required drug dose to a distal tissue.

According to another aspect of the invention, there is provided a method of delivering medication. The method includes positioning one or more stent struts on a luminal surface. In addition, the method includes releasing one or more biologically active compounds intended for local and/or distal delivery from spatially distinct surfaces of the one or more stent struts. Moreover, the method includes determining distinct surfaces not in contact with the luminal surface and such that the released one or more biologically active compounds is convected to distal tissue.

### BRIEF DESCRIPTION OF THE DRAWINGS

FIGS. 1A-1B are a schematic diagrams illustrating various embodiments of the invention;

FIG. 2 is a contour map of drug concentration for a case simulating steady flow coupled with mass transfer;

FIG. 3 is a schematic diagram illustrating a drug delivery system where molecules targeted for local wall delivery or distal, regional delivery depending on the strut surface from which they are released;

FIGS. 4A-4D are schematic diagram illustrating various shaped stent strut structure;

FIGS. 5A-5D are differential concentration profiles when only top, bottom, upstream or downstream stent surfaces are drug-coated, respectively;

FIGS. 6A-6B are schematic diagrams illustrating the recirculation zones produced by various shaped stent strut structures;

FIG. 7A is a schematic diagram illustrating a strut tapering designed to minimize proximal and distal recirculation zones; FIGS. 7B-7D are differential concentration profiles at various surface locations of the strut tapering;

FIG. 8 is a schematic diagram illustrating unapposed device elements with potentially substantial free stream contact used in accordance with the invention;

FIGS. 9A-9B are schematic diagrams illustrating redundant vascular supplies to downstream tissue beds for post-CABG and collateral flow; and

FIGS. 10A-10B are schematic diagrams illustrating endovascular intervention at CABG touchdown sites such that the drug delivery medical system is implanted in a native vessel proximal to the CABG touchdown site and used in accordance with the invention.

### DETAILED DESCRIPTION OF THE INVENTION

The invention provides an endovascular device and techniques which provide controlled delivery of therapeutic agents to local and/or regional targets. For purposes of clarity, the term “local ” is used to describe the vessel wall immediately juxtaposed to an implanted device and regional to describe those vascular beds downstream of the device. In all envisioned cases, at least one active agent will be released for downstream regional targets, while molecules may or may not be targeted for local delivery. This targeting will be achieved through the use of specialized device geometries and spatial patterning of the loaded drugs. It should be noted that a variety of techniques can be used to manufacture such a device and are well-known to those versed in the field.

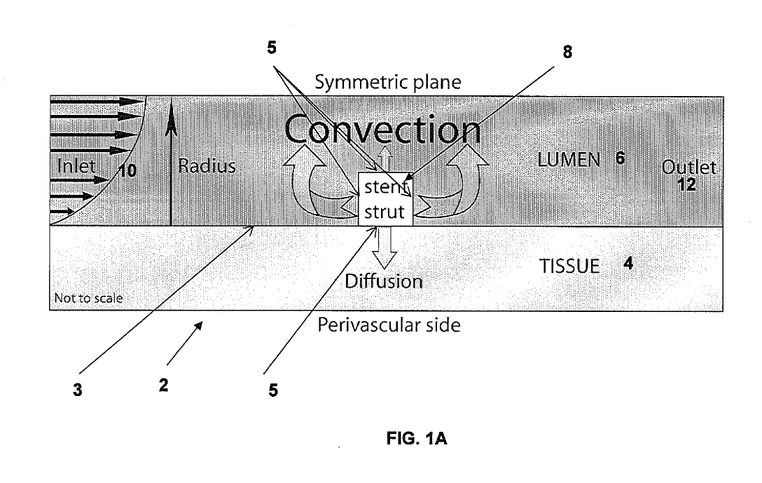

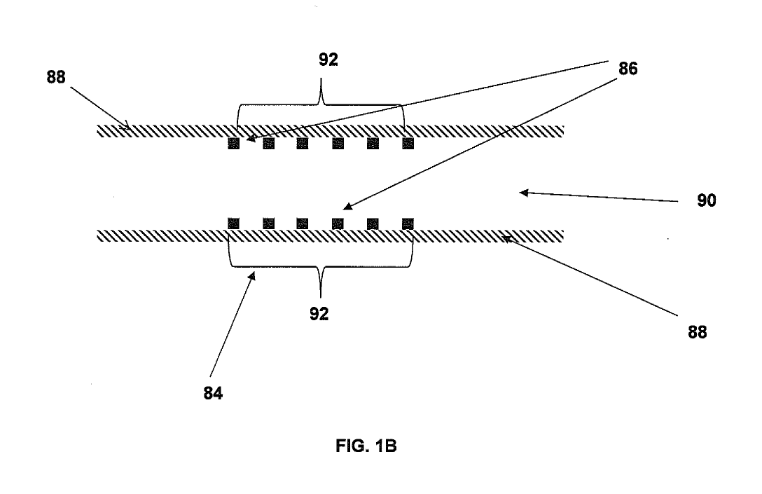

A great deal of effort has been made to understand the factors involved in local drug delivery and targeting and this effort has enabled tremendous evolution in the optimization of local in-stent delivery. Local factors govern local pharmacokinetics such as diffusive and convective forces as well as molecular binding and decay as shown in FIG. 1A. In particular, FIG. 1A shows a drug delivering medical system 2 positioned in a lumen 6 with inlet 10 and outlet 12, where the medical system has a stent strut structure 8 in contact with lumen wall 3. Molecules released from particular stent strut surfaces 5 are partitioned to the lumen 6 and convected downstream to distal tissue 4. The stent strut structure 8 releases at least one biologically active compound intended for distal delivery and providing sufficient surface area for delivering the required drug dose to the distal tissue 4. FIG. 1B shows a drug delivering medical system 84 having stent 92 that includes a plurality of stent struts 86 positioned on vessel walls 88 in a lumen 90 used in accordance with the invention.

Key to the invention is to provide a stent device comprised of distinct strut elements themselves of which have distinct strut element surfaces and to release drug from at least a portion of these surfaces such that a significant portion of drug being released by the drug delivery medical system is released to free-stream flow and convected to tissue distal of the stent. By optifnal design of stent strut element shapes and geometric patterns of the plurality of stent strut elements, surfaces can be created and selected from which released drug is convected to distal tissue.

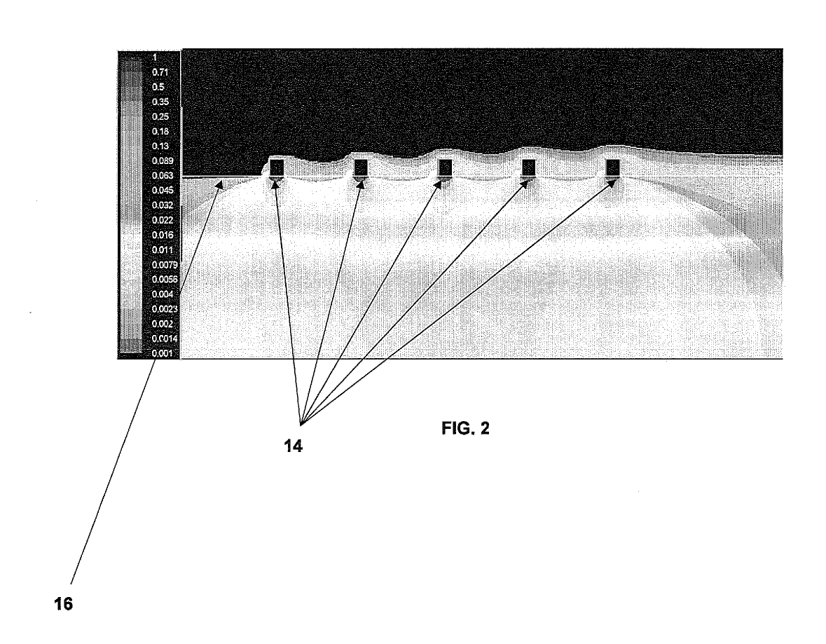

FIG. 2 shows drug concentration contours (log scale) for a case simulating steady flow coupled with mass transfer. A two-dimensional computational domain models drug delivery for 5 stent struts uniformly coated with drug 14 residing at the lumen-tissue interface 16. Abluminal surfaces in conjunction with inter-strut recirculation zones create a uniform drug distribution profile within the tissue. About 26% of the drug is delivered to the free stream. Using such local pharmacokinetic knowledge, tremendous potential exists to optimize regional delivery.

The basis of the inventive approach comes from a basic recognition that a portion of drug is lost to the free stream. Moreover, the invention proposes that this otherwise undesirable form of release or ‘waste’ can be harnessed, controlled and optimized through specific device designs that promote regional delivery distinct from local delivery. The ability to differentially target local and regional sites expands the therapeutic nature of the delivered agents from those that specifically alter local processes (such as platelet/fibrin deposition and smooth muscle cell overgrowth witnessed in in-stent thrombosis and restenosis) to those that have broader impact on regional vascular beds.

For example, agents can be used which alter the progression of natural atherosclerotic disease. They may be growth factors that promote physiologic responses such as angiogenesis or myocyte viability. Alternatively they may be agents such as anti-thrombotic or fibrinolytic compounds capable of breaking up distal embolization or vaso-active agents which counter conditions such as vasospasm. In addition, with the goal of targeting downstream regions, the nature of the device itself can be reformulated. No longer need the device be implanted into regions of stenosis or disease, but potentially into healthy segments of the vessel where delivery of agents to downstream regions would be of benefit.

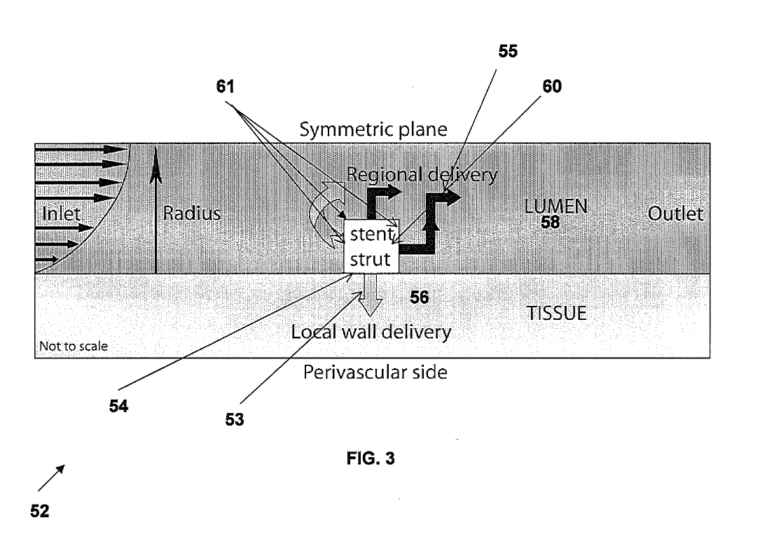

FIG. 3 shows a drug delivery system 52 where molecules targeted for local wall delivery 53 in juxtaposed tissue 56 can be delivered solely from wall contacting surfaces 54 while molecules targeted for distal regional delivery 55 can be released from non-wall-contact surfaces 61 of the stent strut 60 in direct contact with blood in lumen 58, as shown in FIG. 3. Note a multitude of stent struts can be used in the same fashion as described in FIG. 3.

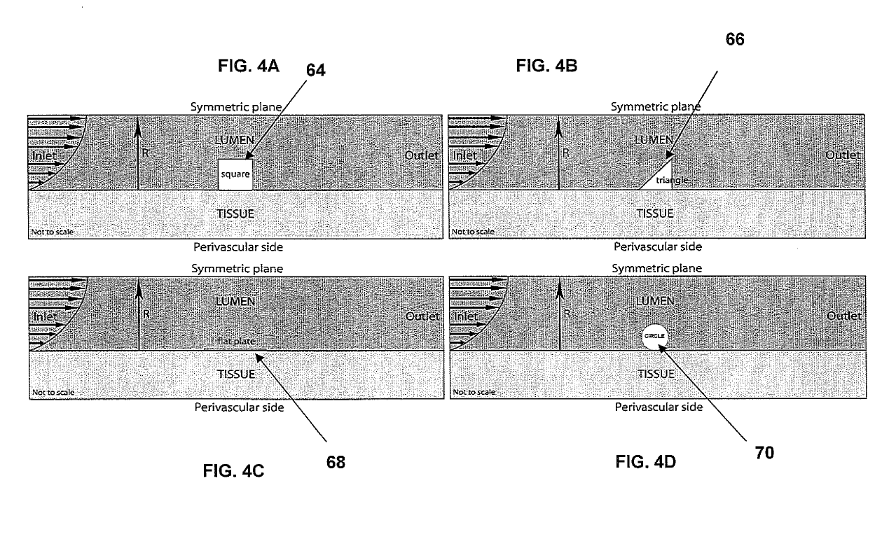

FIGS. 4A-4B illustrate various shaped stent strut structures 64-70 used in accordance with the invention. FIG. 4A shows a stent strut 64 having a square shape while FIG. 4B shows a stent strut 66 having triangular shape. FIG. 4C shows a stent strut 68 comprising a flat plate while FIG. 4D shows a stent strut 70 having a circular shape. The stent strut can also be comprised of various elliptical shapes as well.

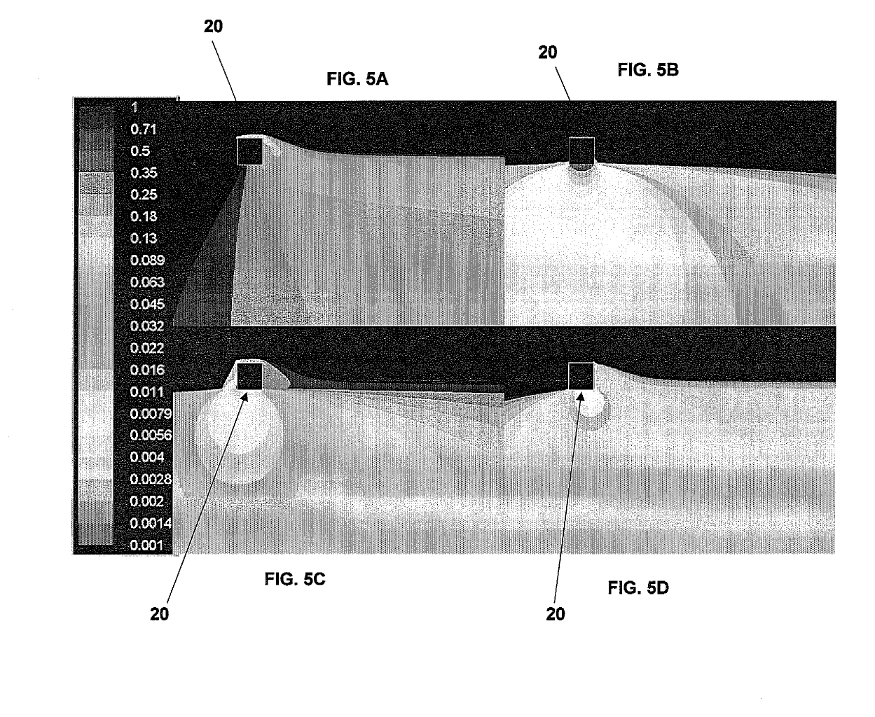

It is known that the particular strut side from which a drug is released greatly affects the pattern of local wall deposition. There are distinct differences when drug is released from the top, bottom, upstream, and/or downstream surfaces when exposed to flow. Local and regional delivery profiles could be significantly controlled when molecules are released from different surfaces of the stent strut. FIGS. 5A-5D show the differential concentration profiles when only (i) top (FIG. 5A), (ii) bottom, (FIG. 5B) (iii) upstream (FIG. 5C) or stent surfaces are drug-coated (FIG. 5D), respectively. For the case shown in FIG. 5A, 43% of the total drug was targeted to the free stream, whereas for case shown in FIG. 5B, only 0.15% was targeted to the free stream. Furthermore, about 12% of the total drug was targeted to the free stream for the case shown in FIG. 5C and about 22% was delivered to the free stream for the case shown in FIG. 5D.

In addition to surfaces with direct strut-wall contact, surfaces that release drug into zones of fluid recirculation upstream and downstream of a particular strut play a key role in governing local drug deposition by essentially sequestering drug and distributing it to inter-strut loci. Thus, surfaces that minimize molecular release via wall contact and into recirculation zones will selectively target the free stream and thus, downstream regional vascular beds. In standard, well studied square strut designs, the optimal surface for distal delivery would be solely the top surface as opposed to all luminal edges.

In addition to optimizing differential delivery by selecting the device surface from which compounds are to be released (i.e. top), some embodiments will alter strut and stent design to further optimize differential targeting to local and regional targets. In some embodiments, struts with upstream and/or downstream tapering will be used to minimize the flow separation induced by corner flow. Sides may be curved as apposed to flat to further create smooth flow transitions. Such designs concurrently minimize recirculation and augment exposed surface area, thus maximizing the surface available for efficient free stream delivery.

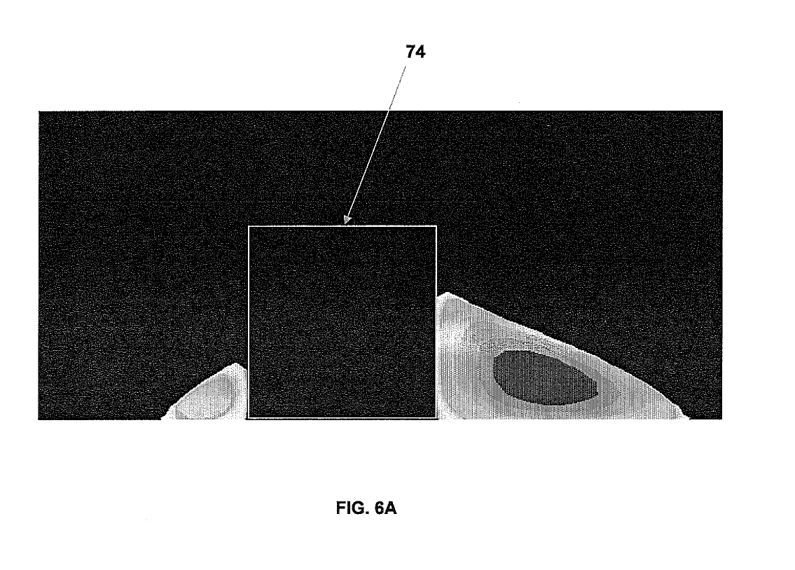

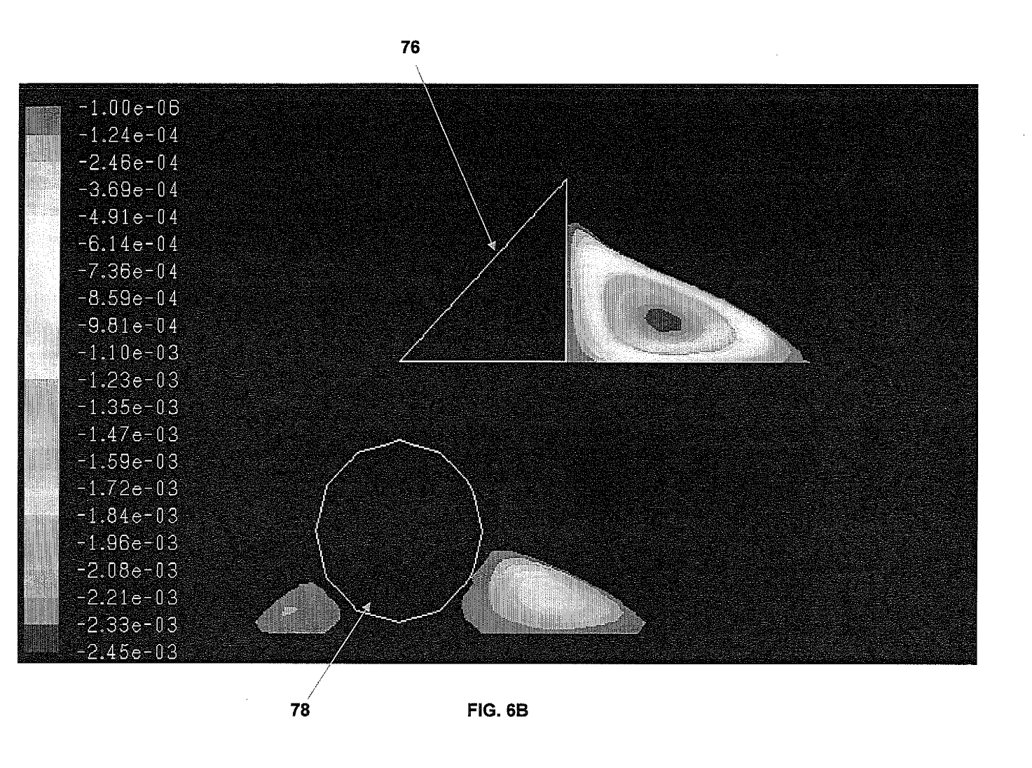

FIG. 6A shows the recirculation zones produced by a square stent strut 74. FIG. 6B shows the recirculation zones produced by a triangular stent strut 76 and pentagonal stent strut 78. These examples, illustrated in FIGS. 6A-6B, show that the extent, number and location of recirculation zones can be clearly controlled by changing the intrinsic shape of the stent strut.

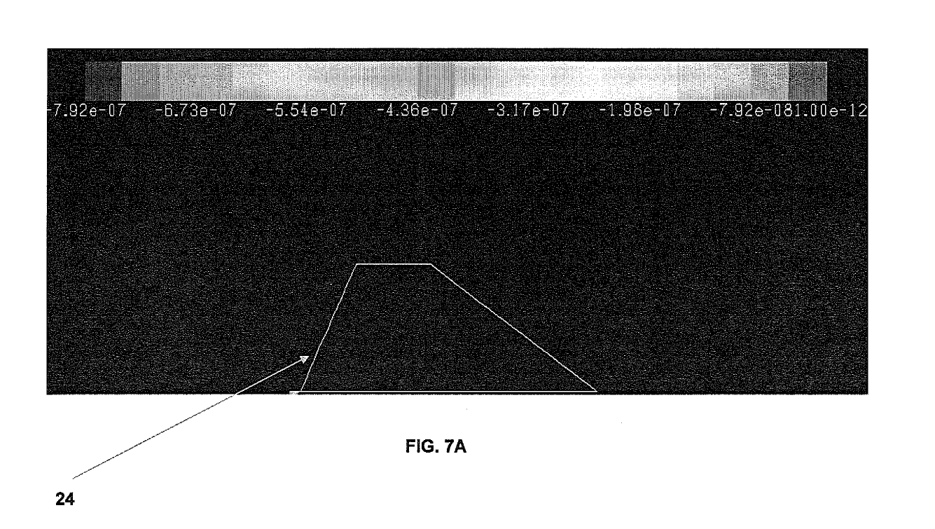

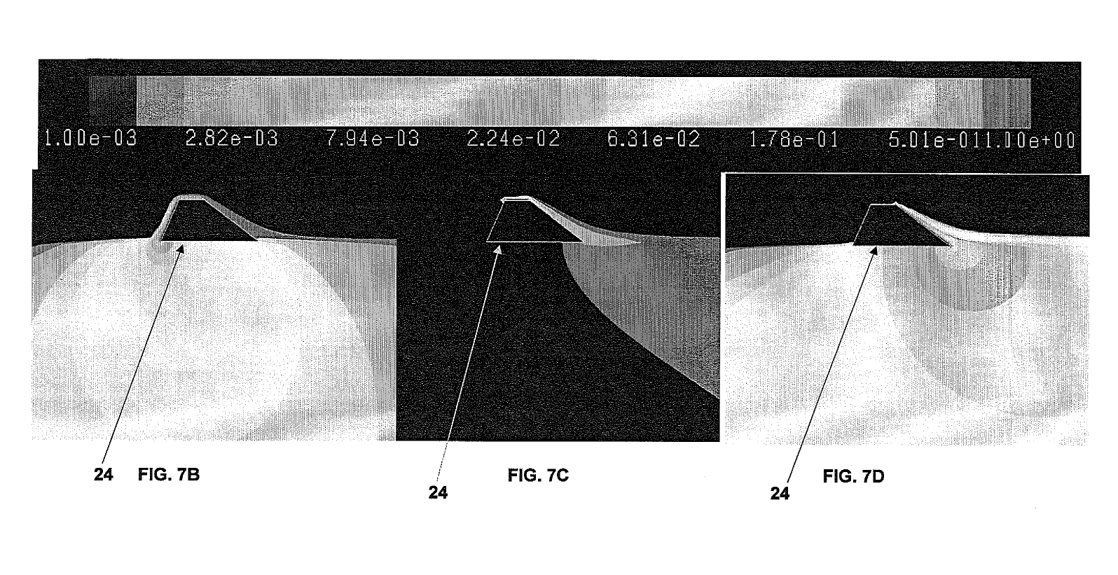

FIG. 7A shows a strut tapering 24 being designed having a trapezoidal shape to minimize proximal and distal recirculation zones. This strut shape 24 provides distinct drug distribution profiles depending on the location of the drug elution source. Drug distribution is depicted for a single drug source at the left as shown in FIG. 7B, right as shown in FIG. 7C, or top strut surfaces as shown in FIG. 7D, respectively for a strut 24 having both upstream and downstream tapering. In comparison to a square strut shown in FIGS. 5A-5D, 41% less drug eluted into the free stream when only the left surface of the tapered strut 24 eluted drug. Also, when the top surface of the strut 24 was only drug eluting, then 46% less drug eluted into the free stream. However, when only the right surface of strut 24 was drug eluting, 148% more drug was targeted into the free stream.

Novel to these embodiments will be the differential ability to release molecules to the vessel wall, and distinctly into the free stream and/or distinctly into zones of recirculation.

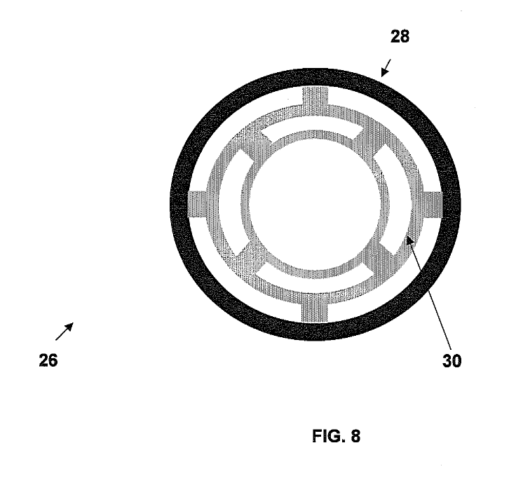

In yet other embodiments, entirely novel stent strut 26 can be used that seek not simply to appose the vessel wall or appose surface 28, but to extend into the luminal flow with “unapposed surfaces” 30 thus dramatically increasing the flow contact surfaces and the potential for free stream delivery, as shown in FIG. 8. A portion of the novel stent strut 26 can be in contact with a vessel wall to anchor position while other potions of stent strut 26 are unapposed and thus in circumferential contact with the free stream. The unapposed surfaces 30 can be biodegradable. The unapposed surfaces 30 form a network that greatly increases the surface area of free stream contact. The stent strut 26 can be expanded into at least two states: one state where there are opposed element surface 28 and unapposed elements 30 and another further expanded state where at least a portion of the unopposed surfaces 30 can be apposed. The unapposed element surfaces 30 are non-thrombotic.

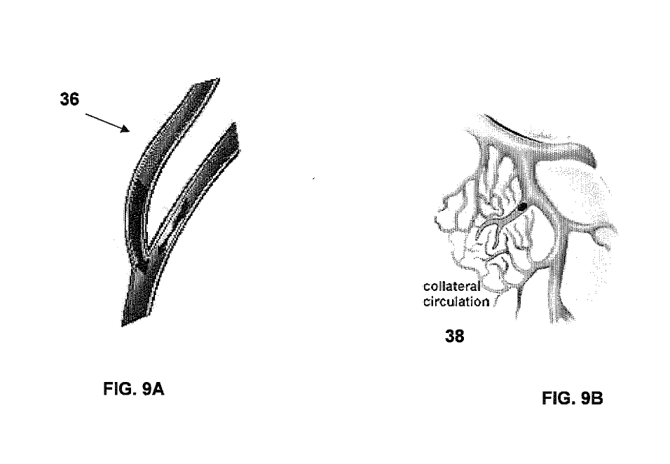

While drug-eluting endovascular devices described in the prior art allow for luminal flow, the presence of significant device elements which extend into the lumen could create a significant resistance to flow and have not been considered a favorable quality. The invention can be designed for use in redundantly supplied vascular beds as in the case of post-coronary artery bypass 36 or collateral vasculature 38, as shown in FIG. 9A-9B respectively. In these instances, devices that are designed to deliver large amounts of luminal drug by offering a large surface area exposed to convective flow can be implanted into one limb of the vascular supply.

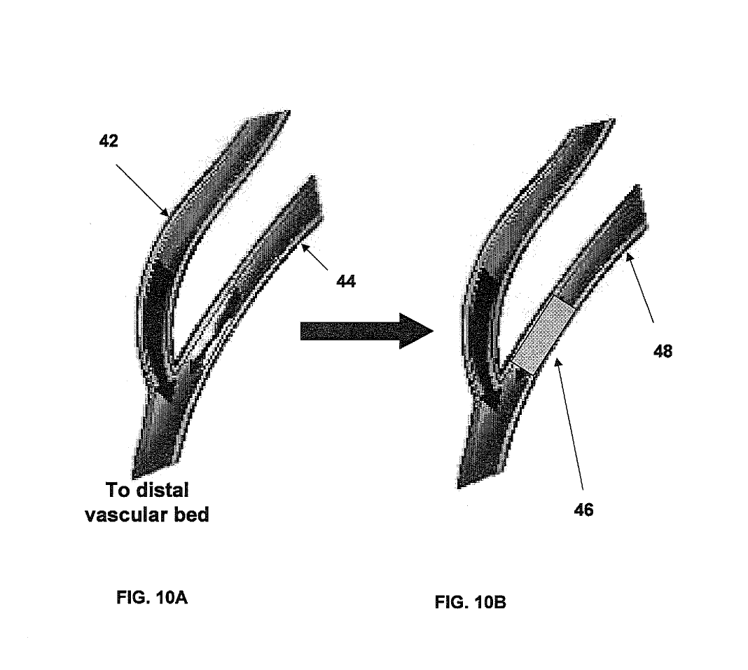

While the invention can significantly impede the luminal flow, the additional limbs can provide the means to deliver blood and nutrients to and from the vascular bed. This embodiment is ideal for use in CABG where bypass grafts 42 are used to bypass diseased, native vessels 44 that supply unhealthy myocardium. While the bypass grafts 42 serve to deliver blood flow, the native vessels 44 are typically left untreated with significant flow limiting occlusion. Opening the native circulation is typically contraindicated in the setting of a widely patent CABG graft to ensure graft maturation, as shown in FIG. 10A. The invention can be designed for luminal delivery to be placed into such native vessels 46 where drug could be delivered to the downstream, diseased beds, while the flow resistance induced by device presence would induce good flow down the graft vessel 48 and graft maturity, as shown in FIG. 10B. The structures can be implanted in native vessels upstream of coronary artery bypass surgery (CABG) graft touch down sites.

Selective regional/local delivery devices would have wide spread application not only in CAD, but in peripheral vascular disease (PAD) as well, and while the invention is based on the great deal of scientific foundation underlying coronary intervention, it is not limited to diseased, coronary vascular bed. The invention can be applied in any location where downstream delivery of molecular agents into distal, tapering vascular beds not readily amenable to stenting or bypass would be of benefit.

Moreover, the invention can be implanted not only at sites of local disease, but can be used in novel therapeutic applications where endovascular devices are implanted into non-stenotic sites and into relatively healthy segments proximal to sites of downstream concern where regional delivery of molecules such as drug or growth factors would be of use. Such applications need not, but may incorporate concomitant local delivery to counter the local effects of device presence. Also, biodegradable backbones of the geometric and release characteristics described herein can further minimize the effect of local device presence allowing a more pure focus on the downstream regional delivery of therapeutic agents.

Although the present invention has been shown and described with respect to several preferred embodiments thereof, various changes, omissions and additions to the form and detail thereof, may be made therein, without departing from the spirit and scope of the invention.

What is claimed is:

1. A drug delivering medical system intended for placement into a blood vessel and comprising a stent device having a plurality of interconnected distinct strut elements comprising distinct strut element surfaces; such that at least some of the distinct strut surfaces are neither in contact with the lumen wall nor in contact with wall-contacting flow recirculation zones, said stent device releasing at least one biologically active compounds intended for distal delivery and providing sufficient surface area for delivering the required drug dose to a distal tissue.

2. The drug delivering medical system in claim 1, wherein the interconnected distinct strut elements are shaped to minimize flow recirculation over the one or more stent struts.

3. The drug delivering medical system of claim 2, wherein the interconnected distinct strut elements comprise trapezoidal elements.

4. The drug delivering medical system of claim 2, wherein the interconnected distinct strut elements comprise elliptical elements.

5. The drug delivering medical system of claim 2, wherein the interconnected distinct strut elements comprise triangular elements.

6. The drug delivering medical system of claim 1, wherein the interconnected distinct strut elements comprise first elements that contact a vessel wall to anchor position while second elements are unapposed and thus in circumferential contact with a free stream.

7. The drug delivering medical system of claim 6, wherein the second elements are biodegradable

8. The drug delivering medical system of claim 6, wherein the second elements form a network that greatly increases the surface area of free stream contact.

9. The drug delivering medical system of claim 6, wherein the second elements are non-thrombotic.

10. The drug delivering medical system of claim 6, wherein the interconnected distinct strut elements induce a flow resistance to enable maturation of a coronary artery bypass graft that touches down distal to its position.

11. A method of delivering medication comprising:
positioning one or more stent struts on a luminal surface;
releasing one or more biologically active compounds intended for local and/or distal delivery from spatially distinct surfaces of one or more stent struts; and
determining distinct surfaces not in contact with said luminal surface and such that said released one or more biologically active compounds is convected to distal tissue.

12. The method in claim 11, wherein the one or more stent struts are shaped to minimize flow recirculation over the one or more stent struts and distally delivered drug is released into luminal surfaces that are not in contact with wall-contacting recirculant flow.

13. The method of claim 12, wherein the one or more stent struts comprise trapezoidal elements.

14. The method of claim 12, wherein the one or more stent struts comprise eliptical elements.

15. The method of claim 12, wherein the one or more stent struts comprise triangular elements.

16. The method of claim 11, wherein one or more stent struts comprise first elements that contact a vessel wall to anchor position while second elements are unapposed and thus in circumferential contact with a free stream.

17. The method of claim 16, wherein the second elements are biodegradable

18. The method of claim 16, wherein the second elements form a network that greatly increases the surface area of free stream contact.

19. The method of claim 16, wherein one or more stent struts is expanded into at least two states: one state where there are first elements and second elements and another further expanded state where at least a portion of the second elements are apposed.

20. The method of claim 16, wherein the second elements are non-thrombotic.

21. The method of claim 16, wherein the one or more stent struts induce a flow resistance to enable maturation of a coronary artery bypass graft that touches down distal to its position.
